(19) United States
(12) Patent Application Publication
(10) Pub. No.: US 2024/0366154 A1
(43) Pub. Date: Nov. 7, 2024

(54) INTERACTIVE PATIENT MONITORING SYSTEM

(71) Applicant: Cigna Intellectual Property, Inc., Wilmington, DE (US)

(72) Inventors: Tara W. Esse, Boerne, TX (US); Lane Ramirez, Phoenix, AZ (US)

(21) Appl. No.: 18/141,526

(22) Filed: May 1, 2023

(51) Int. Cl. A61B 5/00; A61B 5/145; A63H 11/20

(52) U.S. Cl. CPC A61B 5/6896; A61B 5/14532; A61B 5/7267; A61B 5/7275; A61B 5/7282; A61B 5/741; A61B 5/747; A63H 11/20; A63H 2200/00

(57) ABSTRACT

An interactive patient monitoring system is disclosed including a robotic apparatus with physical characteristics representative of an animal or other companion with whom a human could interact, and one or more actuators capable of moving the robotic apparatus; a communication device configured to communicate with a sensing device to receive measurements of one or more medical characteristics of a patient; and a controller configured to examine the measurements received from the sensing device and to control the one or more actuators of the robotic apparatus to interact with the patient based on the measurements received from the sensing device.

### BACKGROUND

Individuals with any variety of medical conditions may require continuous or at least frequent monitoring of their medical condition to remain healthy. For example, an individual with diabetes may need to check their blood glucose levels several times during the day to ensure they do not enter a hypoglycemic or hyperglycemic state. Similarly, individuals with heart conditions may need to routinely monitor their blood pressure, heart rate and/or similar physical parameter.

The monitoring of a medical condition may be comprised of varied degrees of physical involvement. For example, many persons with diabetes who are on medications for diabetes management may prick their skin (e.g., fingers) several times throughout the day to obtain a drop of blood for use with a glucose measuring device to determine their blood glucose level. This monitoring of the blood glucose levels can occur pre-meal, post-meal, and in the course of exercise and throughout illness (as examples), and is critical to ensure the vitality of the person with diabetes. The American Diabetes Association has established goals for pre- and post-meal blood glucose levels for persons with diabetes (reference: Monitoring Your Blood Sugar (cdc.gov) available at https://www.cdc.gov/diabetes/managing/managing-blood-sugar/bloodglucosemonitoring html), and these goals, in addition to other factors, guide providers and care teams regarding recommended frequency of blood sugar monitoring. The frequency of blood glucose checks can become quite cumbersome for many persons with diabetes. An alternative to the repeated finger pricks may include a continuous glucose monitoring device. These continuous glucose monitors (“CGM”) are small devices that are attached to the body, typically the arm or belly of the patient. The CGM device contains a small sensor that is inserted under the skin and measures the glucose levels in the fluid surrounding the cells (i.e., interstitial fluid), thus eliminating the need for painful skin pricks.

A CGM device offers advantages over the more traditional finger prick method. For instance, many persons with diabetes find that repeated pricking of the fingertip becomes painful. Some patients are uncomfortable with the sight of blood or have anxiety about the procedure. A CGM device also allows the patient to better control their glucose levels throughout the day, and by having access to nearly real-time results, to thus avoid hyperglycemic or hypoglycemic conditions. The CGM device can test the glucose level every few minutes and send information wirelessly to a monitor. The monitor may be part of an insulin pump or a separate device carried by the patient. In some cases, a mobile phone or tablet may serve as the monitor. The monitor may provide a numerical reading display and/or be equipped with audible alarms to warn the patient in the event that the blood glucose reading is becoming too high or too low. Some CGM devices allow parents and/or caregivers to monitor their loved one(s) with diabetes while asleep or away from home. Providers, too, may receive notifications of patient blood glucose readings in near real-time, or simply review historical information from the patient's CGM device at a follow-up appointment.

The ability for a person with diabetes or their caregiver to self-monitor and/or make medication dosage adjustments with provider oversight can be compromised if cognitive decline, dementia, or sensory impairment is evident. In particular, a patient with hearing loss may not hear an alarm. Similarly, patients with cognitive decline or dementia may hear the alarm, but may not understand what it means and/or what actions to take to correct the blood sugar, or they may simply forget to check their readings on the monitor.

### BRIEF DESCRIPTION

In one embodiment, an interactive patient monitoring system includes a robotic apparatus having one or more actuators capable of moving the robotic apparatus; a communication device configured to communicate with a sensing device to receive measurements of one or more medical characteristics of a patient; and a controller configured to examine the measurements received from the sensing device and to control the one or more actuators of the robotic apparatus to interact with the patient based on the measurements received from the sensing device.

In one embodiment, a method includes receiving measurements of one or more medical characteristics of a patient from a sensing device, the measurements received at a communication device of a robotic apparatus; identifying a medical state based on the measurements that are received; and controlling one or more actuators of the robotic apparatus to cause the robotic apparatus to interact with the patient based on the medical state that is identified.

In one embodiment, an interactive patient monitoring system includes a robotic apparatus having a shape of an animal pet, the robotic apparatus comprising one or more actuators configured to simulate movement of the animal pet by moving the robotic apparatus; a communication device configured to digitally communicate with a continuous glucose measurement (CGM) device to receive measurements of blood glucose levels of a patient; and a controller configured to examine the measurements received from the CGM device and to control the one or more actuators of the robotic apparatus to simulate movement of the animal pet and interact with the patient in different ways based on the blood glucose measurements received from the CGM device.

### BRIEF DESCRIPTION OF THE DRAWINGS

FIG. 1 is a schematic illustration of an embodiment of the interactive patient monitoring system showing a plurality of actuators internal to the robotic apparatus.

FIG. 2 is a schematic illustration of another embodiment of the interactive patient monitoring system showing a plurality of sensing devices.

FIG. 3 is a schematic illustration of another embodiment of the interactive patient monitoring system showing a plurality of sensing devices and a plurality of actuators both internal and external to the robotic apparatus.

FIG. 4 illustrates a functional block diagram of an example artificial neural network that can be used by the controller for evaluating data or measurements from the sensing devices and determining how to act or operate to assist the patient based on that data or measurements.

FIG. 5 illustrates one example of a robotic apparatus.

In the drawings, reference numbers may be reused to identify similar and/or identical elements.

### DETAILED DESCRIPTION

In one embodiment, an interactive patient monitoring system includes a robotic apparatus having one or more than one actuators capable of moving the robotic apparatus. The robotic apparatus may be configured in the shape of an animal, such as an animal pet like a dog, cat, bird, or other animal. In one embodiment, the robotic apparatus may be configured in a more humanoid shape to resemble a person. In one embodiment, the robotic apparatus may be configured to resemble a mechanical device, such as a robot. The particular physical configuration of the robotic apparatus may be selected to evoke higher levels of interaction with the patient. By way of example, a robotic apparatus configured in the shape of a cat may not be the best option for a patient that is not particularly fond of cats.

In one embodiment, the robotic apparatus has one or more than one actuators to facilitate various actions by the robotic apparatus. The number of actions to be undertaken by the robotic apparatus will be determined by the amount and type of interactions desired between the robotic apparatus and the patient and overall functionality desired for the robotic apparatus. In one embodiment, the actuators may include motors to cause movement by the robotic apparatus or parts of the robotic apparatus. By way of example, such actuators may facilitate movement of the arms, hands, legs, feet, head, tail, neck, wheels, or treads of the robotic apparatus, if so equipped.

In another embodiment, the actuators may facilitate actions to be taken by the robotic apparatus. For example, the robotic apparatus may be equipped with actuators that cause emission of sounds, such as alarms, music, animal noises (bark, meow, purr, whimper) and/or voice commands. In one embodiment, the actuators may cause the robotic apparatus to emit light or vibrate, which may be particularly useful for patients who are visually or hearing impaired. Some actuators may cause movement of the robotic apparatus by actuating, controlling, powering, etc., motors, while other actuators may cause other actions of the robotic apparatus by actuating, controlling, powering, etc. other devices that do not move the robotic apparatus (e.g., lamps, speakers, cellular transceivers, etc.).

In a further embodiment, the actuators may enable the robotic apparatus to initiate a phone call to a pre-determined recipient, such as a relative or caregiver, a physician's office or emergency medical personnel. Such an initiation may only occur when, for example, blood glucose levels are outside of a pre-determined range or set of triaged ranges (e.g., if slightly elevated or below range, a loved one is notified; if substantially elevated or below range, emergency personnel is alerted). Such calls may, for example, be initiated by the robotic apparatus or may be the result of the robotic apparatus directing another device, such as an in-home, always on, smart speaker virtual assistant like the Echo®/Alexa® systems from Amazon, to make the call. In one embodiment, the robotic apparatus may be equipped with actuators that enable the robotic apparatus to communicate with other devices in a smart home, such as lights, door locks, automatic garage doors, thermostats, alarm systems, doorbells, security cameras and the like when, for example, the blood glucose levels are outside of a pre-determined range. Similarly, the robotic apparatus may include actuators to effect data transmission, such as raw data from the sensors, to a health care provider, a family member, an insurance provider, or to a central location.

In a particular illustrative embodiment, the robotic apparatus may be equipped with an acoustic output device capable of generating different sounds, including different voices and an acoustic input device capable of detecting the voice of the patient. In one example, the voice may be a recorded default voice that is the same or common to multiple robotic apparatuses. Optionally, the voice may be a recorded voice of a selected individual, such as a family member, a caregiver, a celebrity, or the like. The recorded voice of a person that is familiar to a patient may be more effective in communicating with the patient, especially where the patient may be suffering from neurological disorders such as dementia. In some embodiments, the sounds may be generated from a library of pre-stored voice recordings. For example, the voice recordings may be a patient's mother indicating that the patient has a blood sugar level at an unacceptable level and that certain action should be taken. In some embodiments, the sounds may be synthesized from a library of sounds recorded on behalf of the patient by a person or a set of persons. In some embodiments, artificial intelligence may be used to generate the sounds based on a discrete sound recording data set of a person, based on voices recorded by the robotic apparatus, or otherwise. A controller (described below) would be able to control the acoustic input device, the acoustic output device, and the communication device (described below) to audibly connect the patient with a third party via the robotic apparatus.

In yet another embodiment, the interactive patient monitoring system may include one or more sensing devices that monitor the medical or physical conditions of the patient. The sensing device may be attached to the body of the patient or made a part of the robotic apparatus. Examples of such sensing devices may include blood glucose monitors, pacemakers, heartbeat monitors, blood pressure monitors, infrared or other sensors to monitor body temperature, moisture sensors to monitor sweat, olfactory sensors to monitor scents and smells, spatial recognition sensors to monitor physical posture and location, and acoustic sensors to monitor speech patterns, speech quality and/or volume.

With respect to olfactory sensors, these types of sensors can include electronic noses, chemosensors, gas chromatography sensors, or the like. These sensors can detect different types of smells with some of the smells being indicative of a medical condition or event. For example, some studies have shown that Parkinson's disease can create an odor that may be detected by the sensors, that diabetics undergoing hypoglycemic or hyperglycemic events may produce an odor that can be detected by the sensors, etc.

Another example of a sensing device can include one or more optical sensors, such as cameras, infrared detectors, or the like. These sensing devices can obtain video, images, infrared maps, etc., that can indicate whether the patient is exhibiting signs of a medical issue. For example, the sensing devices can detect whether the patient's pupils are dilated or constricted, whether the patient is sweating, whether the patient is shaking, etc. These signs can indicate a hypoglycemic event as one example.

With respect to speech patterns, the controller can examine speech patterns of the patient to decide whether the speech patterns or changes in the speech patterns of the patient indicate a medical event. For example, during a hypoglycemic event, the speech of a patient may become slurred or unintelligible. The controller can monitor and compare recorded sounds of the patient speaking to determine whether the patient's speech has changed and may indicate a medical event.

Depending on the type of sensing device used and their intended purpose, the sensing devices may be placed on the body of the individual patient or made as a component of the robotic apparatus, or both.

The one or more than one sensing devices are in operative communication with a communication device, which is configured to receive data and information from the one or more than one sensing devices. As used herein, the terms “communication” and “communicate” refer to the receipt or transfer of one or more than one signals, messages, commands, or other type of data. For one unit or component to be in communication with another unit or component means that the one unit or component is able to directly or indirectly receive data from and/or transmit data to the other unit or component. This can refer to a direct or indirect connection that may be wired and/or wireless in nature. Additionally, two units or components may be in communication with each other even though the data transmitted may be modified, processed, routed, and the like, between the first and second unit or component. For example, a first unit may be in communication with a second unit even though the first unit passively receives data, and does not actively transmit data to the second unit. As another example, a first unit may be in communication with a second unit if an intermediary unit processes data from one unit and transmits processed data to the second unit. It will be appreciated that numerous other arrangements are possible.

The sensing devices and communication device may be communicatively coupled to one or more than one networks. These networks can be wireless networks, such as networks that communicate signals between wireless communication devices, such as antennas, satellites, routers, etc. In some embodiments where a wireless network is utilized, it may be desirable to include one or more wireless signal amplifiers or boosters to ensure adequate communication between the one or more sensing devices and the communication device throughout the patient's environment even if the patient is spatially separated from the robotic apparatus. Alternatively, the network can be a hard-wired network, or a network that utilizes cellular or landline phone transmission.

The communication device is in operative communication with a controller. The controller analyses the information obtained by the communication device, determines the appropriate course of action, and then activates one or more of the actuators to cause a desired activity by the robotic apparatus. The controller represents hardware circuitry that includes and/or is connected with one or more processors (e.g., micro-processors, controllers, field programmable gate arrays, and or integrated circuits). In some embodiments, the controller includes and/or is connected to a data storage/memory device, such as a computer hard drive, a CD-ROM drive, a DVD-ROM drive, a removable memory card or USB stick, a magnetic tape, or similar device. In some embodiments, the controller device may include and/or be connected to an analysis processing unit which could examine images, infrared and thermal data, and other input data from the one or more sensing devices. The analysis processing unit can include or represent one or more the one hardware circuits or circuitry that includes and/or may be coupled with one or more than one computer processors (e.g., microprocessors) or other electronic logic-based devices.

The controller receives the input data from the communication device and examines the data. The examination can include comparing an input data point to one or more than one previously acquired data points (such as to identify a trend), comparing the data point to a baseline data point (such as one representing a resting heart rate), and combining an input data point with one or more than one other input data points from the sensing units. Using stored baseline data points, artificial intelligence and machine learning, the controller identifies what actions needs to be taken by the robotic apparatus and also what actions are not to be taken based on all input data received from the sensing devices through the communication device, and signals the robotic apparatus to act accordingly via the one or more than one actuators. The controller may include or be part of a machine learning or artificial intelligence neural network configured to examine the measurements received from the sensing device using a model that defines relationships between different actions of the robotic apparatus and different values or trends of the measurements and may be configured to select one or more than one of the different actions and to control the one or more than one of the actuators to implement the one or more than one of the different actions that is selected using the model.

In some embodiments, the controller may be configured to act based on the initial measurements received from the sensing devices, or may be configured to act on secondary input from one or more internal sensors of the robotic apparatus that sense a second measurement of a medical characteristic of the patient. For example, if the sensing device transmits an initial blood glucose reading, the controller may not cause the robotic apparatus to act. However, if the internal sensors of the robotic apparatus also detect a high level of sweat, or a drop in body temperature, then the controller may cause the robotic apparatus to act. These sensors may include infrared detectors that sense temperature from a distance, cameras that obtain images or video (which can be analyzed by the controller to determine whether the person's face appears to be wet or damp from perspiration, whether the person is shaking, trembling, or stumbling, etc., due to a potential hypoglycemic event or other medical event), or the like.

The controller may examine data from different sensors and/or different types of sensors to rule out other potential causes for detected events. For example, a person who has just exercised and a person undergoing a hypoglycemic event may both appear to be wet or damp with perspiration. The controller may not rely solely on the sensor that detected the perspiration (e.g., the camera), but can evaluate other biometric parameters or data, such as output from a continuous glucose meter, an infrared sensor, a heart rate monitor, or the like. Examining different types of data from different types of sensors can be used to identify and differentiate medical events from other types of events.

The controller may automatically (e.g., without intervention by the patient or another person) contact family, emergency personnel, or healthcare personnel responsive to the data or measurements from the sensing device indicating the patient's need for assistance. For example, one or more of the sensing devices of the robotic apparatus may include a global navigation satellite system (GNSS) receiver (such as a global positioning system (GPS) receiver) that determines a location of the robotic apparatus, a wireless transceiver that can use wireless triangulation to determine the location of the robotic apparatus, a cellular transceiver that can use wireless triangulation to determine the location of the robotic apparatus, or the like. Responsive to determining that the patient may be in need of assistance, the robotic apparatus can communicate a signal (e.g., a telephone call, an email, a text message, or another type of message) to a family member, designated caregiver, doctor, emergency personnel, or the like. This signal can provide the location of the patient (via the location of the robotic apparatus), and optionally can indicate the problem or event occurring in which the patient may need assistance.

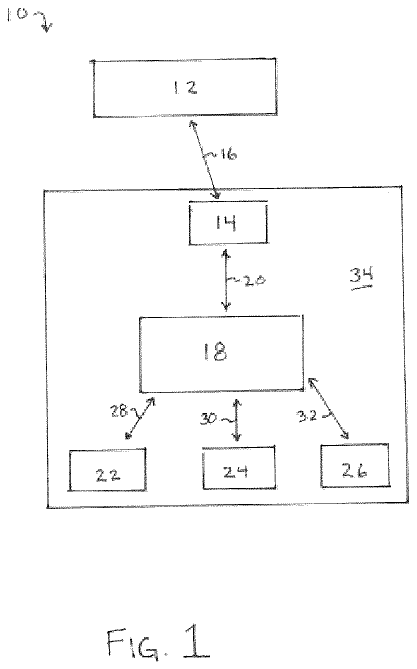

With reference now made to FIG. 1, a schematic illustration of an embodiment of the interactive patient monitoring system is shown therein. In the illustrated embodiment, the interactive patient monitoring system 10 includes a sensing device 12 which gathers and transmits patient data to a communication device 14. The communication device 14 and the sensing device 12 are in operative communication with one another via a communication channel as illustrated by the double-headed arrow 16. Information received by the communication device 14 is in operative communication with the controller 18 via communication channel 20.

The controller 18 analyzes the information received from the communication device and determines whether some action is required and, if so, the appropriate action to take. For example, based on the information received, the controller 18 may determine to seek additional information from the sensing device 12, such as directing the sensing device 12, via the communication device 14, to take an addition measurement of patient data. The controller 18 is in operative communication with one or more actuators 22, 24, 26 via respective communication channels 28, 30, 32 and is thus able to cause an appropriate action to be taken by a robotic apparatus 34 as described herein.

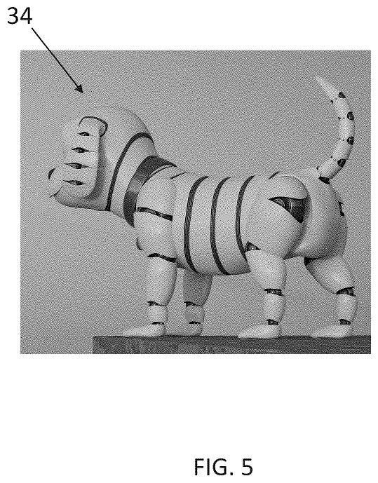

FIG. 5 illustrates one example of the robotic apparatus 34. While the illustrated robotic apparatus 34 is shown as a robotic canine or dog, not all embodiments of the inventive subject matter are limited to the robotic apparatus 34 having the appearance or shape of a canine or dog. For example, in another embodiment, the robotic apparatus 34 may have the shape and/or appearance of a feline or cat, of a bird (e.g., parrot), a humanoid shape and/or appearance, or any other shape and/or appearance with whom a human could interact.

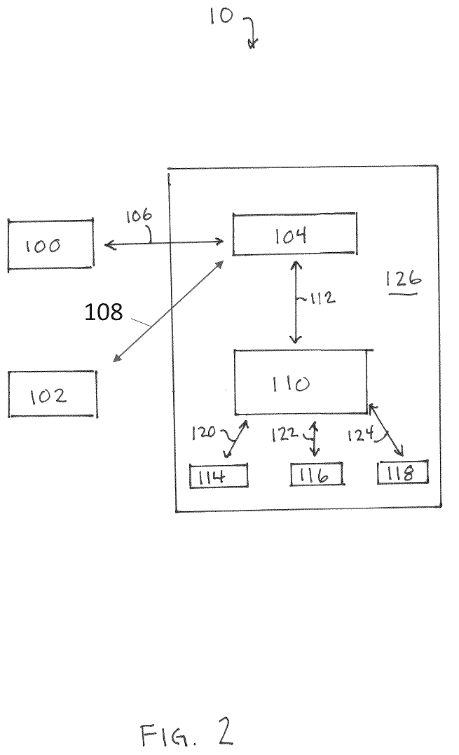

With particular reference to FIG. 2, another embodiment of the interactive patient monitoring system 10 is schematically illustrated therein. In this embodiment, a plurality of sensing devices 100, 102, are shown in operative communication with a communication device 104 via respective communication channels 106, 108. As in the prior embodiment shown in FIG. 1, the communication device 104 is in operative communication with controller 110 via communication channel 112. The controller 110, in turn, is in operative communication with one or more actuators 114, 116, 118 via respective communication channels 120, 122, 124 and thus is able to control movement or other actions of the robotic apparatus 126.

As previously explained, the sensing devices 100, 102 may be external to a patient and may, for example, be a motion sensor to determine if a patient is ambulatory, an ambient temperature sensor, an auditory sensor to determine sounds, an optical device to obtain images or videos of the patient, and similar devices that are not directly transmitting information about a patient's medical information, but may nevertheless be important in providing the controller 110 with all relevant information needed to determine an appropriate course of action for the robotic apparatus 126.

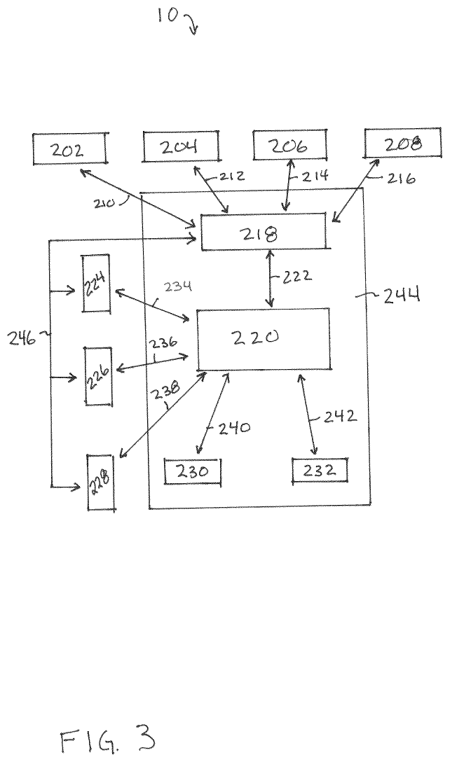

FIG. 3 is schematic illustration of yet another embodiment of the interactive patient monitoring system 10. In this particular embodiment, the interactive patient monitoring system 10 includes a plurality of sensing devices 202, 204, 206, 208 each having a respective communication channel 210, 212, 214, 216 whereby the sensing devices are in operative communication with a communication device 218. A controller 220 is in operative communication with the communication device 218 via communication channel 222. In this particular embodiment, a plurality of actuators 224, 226, 228, 230, 232 are in operative communication with the controller 220 via respective communication channels 234, 236, 238, 240, 242. As seen in FIG. 3, the actuators may be internal to the robotic apparatus 244 or may be external, as in the case of actuators 224, 226 and 228. Examples of these external actuators may include devices in a smart home, including garage door openers, doorbell cameras, light switches, thermostats, alarm systems and virtual assistants. The actuators may be in direct communication with the controller 220 as in the case of actuators 230, 232 via communication channels 240, 242 or indirect communication via the communication channel 246 between the communication device 218 and the actuators 224, 226, 228.

By way of non-limiting example, in one embodiment the robotic apparatus may include physical characteristics representative of an animal (such as a dog) or other companion with whom a human could interact, and the interactive patient monitoring system may be used with a diabetic patient with a CGM device. In this non-limiting example where the robotic apparatus is a robotic dog, if the readings from the CGM device indicate to the controller that the patient is in, or trending toward a hypoglycemic state, the controller can cause the robotic dog to bark aggressively and move toward the kitchen area as a way of warning the patient to eat something, drink juice, or take other appropriate action.

In another non-limiting example, in one embodiment the robotic apparatus may include physical characteristics representative of an animal (such as a dog) and the interactive patient monitoring system may be used with a diabetic patient with a CGM device. In this non-limiting example where the robotic apparatus is a robotic dog, if the readings from the CGM device indicate to the controller that the patient is in, or trending toward, a hyperglycemic state, the controller may cause the robotic dog to whimper and move toward the entryway door as a way of reminding the patient to take a walk or engage in exercise.

In yet another non-limiting embodiment, the robotic apparatus may include physical characteristics representative of an animal (such as a cat) and the interactive patient monitoring system may be used with a diabetic patient with a CGM device who is also cognitively impaired. In this non-limiting example where the robotic apparatus is a robotic cat, if the readings from the CGM device indicate to the controller that the patient is in, or trending toward a hyperglycemic state, the controller can cause the robotic cat to meow and scratch at the front door to remind the patient to talk a walk or engage in other exercise. If motion sensors placed on the robotic cat or in the patient environment detect a lack of movement by the patient, the controller can cause the robotic cat to meow aggressively and paw at the legs of the patient as a further attempt to prompt the patient to engage in exercise. If the motion sensors continue to show a lack of movement by the patient, the controller can cause the robotic cat to take increasingly more advanced activities to stimulate the patient into movement. In some embodiments the controller may activate doorbell of the home, or open the garage door as a way to get the patient up and moving.

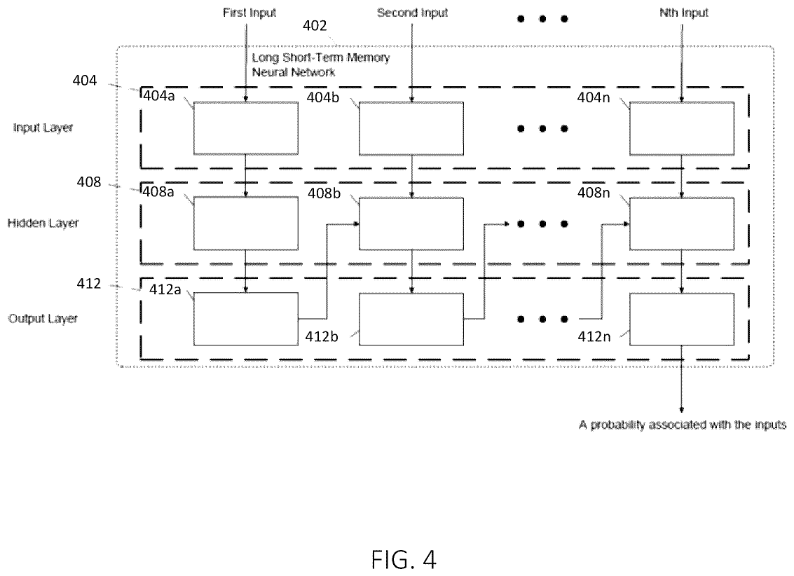

FIG. 4 illustrates a functional block diagram of an example artificial neural network 402 that can be used by the controller for evaluating data or measurements from the sensing devices (e.g., biometric parameters) and determining how to act or operate to assist the patient based on that data or measurements. The neural network 402 can represent a machine learning or artificial intelligence system. For example, the neural network 402 can represent a long short-term memory (LSTM) neural network. In an example, the neural network 402 can represent one or more recurrent neural networks (RNN). The neural network 402 may be used to implement the machine learning as described herein, and various implementations may use other types of machine learning networks. The neural network 402 includes an input layer 404, one or more intermediate or hidden layers 408, and an output layer 412. Each layer 404, 408, 412 includes artificial intelligence units, or neurons. Each neuron can receive information (e.g., as input into the neural network 402 or as received as output from another neuron in another layer or the same layer), process this information to generate output, and provide the output to another neuron or as output of the neural network 402. The input layer 404 may include several input neurons 404a, 404b . . . 404n, the hidden layer 408 may include several intermediate neurons 408a, 408b . . . 408n, and the output layer 412 may include several output neurons outputs 412a, 412b . . . 412n. The inputs may include, for example, data measured by the sensing devices or other information described herein.

Each neuron can receive an input from another neuron and output a value to the corresponding output to another neuron (e.g., in the output layer 412 or another layer). For example, the intermediate neuron 408a can receive an input from the input neuron 404a and output a value to the output neuron 412a. Each neuron may receive an output of a previous neuron as an input. For example, the intermediate neuron 408b may receive input from the input neuron 404b and the output neuron 412a. The outputs of the neurons may be fed forward to another neuron in the same or different intermediate layer 408.

The processing performed by the neurons may vary based on the neuron, but can include the application of the various rules or criteria described herein to partially or entirely decide whether to identify a medical event involving the patient and/or a responsive action to take to assist the patient. The output of the application of the rule or criteria can be passed to another neuron as input to that neuron. One or more neurons in the intermediate and/or output layers 408, 412 can identify the medical event or the responsive action to take to assist the patient. The last output neuron 412n in the output layer 412 may output a signal indicating the identified event or action to take. For example, the output from the neural network 402 can be an identification of a hypoglycemic event, a responsive action to prompt the patient to take a walk or otherwise exercise, a responsive action to play a recording of a healthcare provider or family member to prompt action by the patient, a responsive action to call emergency personnel to help the patient, or the like. Alternatively, the output can be a probability indicating the potential effectiveness of the different responsive actions and/or the potential accuracy of potential medical events. Although the input layer 404, the intermediate layer(s) 408, and the output layer 412 are depicted as each including three artificial neurons, one or more of these layers may contain more or fewer artificial neurons. The neurons can include or apply one or more adjustable parameters, weights, rules, criteria, or the like, as described herein, to perform the processing by that neuron.

The neural network can be trained by operators, automatically self-trained, or can be trained both by operators and self-trained to improve how decisions are made based on the biometric parameters that are measured or received. This can allow for the controller to improve the accuracy with which medical events are identified and/or which responsive actions are selected for the robotic apparatus to perform. For example, given data from various sensors, the neural network may decide that a monitored person is undergoing a hypoglycemic event. This decision can be made by the different neurons examining different portions of the data (e.g., camera images showing the person sweating, infrared temperature measurements showing elevated temperatures, blood glucose measurements, etc.), passing decisions made based on this data to other neurons based on relationships between the neurons (e.g., a group of pixels in the images show perspiration, the measured temperature is warmer than a threshold, and the measured blood glucose is at the low end of a threshold range of values), the other neurons making further decisions, and so on, until the neural network identifies a hypoglycemic event and/or selects a responsive action, such as causing the robotic apparatus to move over to the person, nudge the person, and use a speaker to direct the person to consume glucose tablets.

Feedback can be obtained and provided back to the neural network based on the identified event and/or selected actions. For example, if the person was not actually having a hypoglycemic event, data indicative of this misidentification can be provided to the neural network to change how some of the neurons make decisions on the same or similar data and/or the relationship between some of the neurons (e.g., a neuron identifying perspiration may pass this identification to a different neuron that can examine additional data to confirm or refute this identification). As another example, if the person was having a hypoglycemic event but the selected action was insufficient (e.g., the hypoglycemic event was severe and consuming glucose tablets was insufficient to end the hypoglycemic event), then the neurons can be re-trained to select another action (e.g., call a relative or healthcare provider). This process can be repeated many times in an iterative way so that the neural network repeatedly learns and improves how medical events are identified and/or which responsive actions are selected when the same or similar data is received again.

The above description is illustrative and not restrictive. For example, the above-described embodiments (and/or examples thereof) may be used in combination with each other. In addition, many modifications may be made to adapt a particular situation to the teachings of the inventive subject matter without departing from its scope. Many other embodiments will be apparent to one of ordinary skill in the art upon reviewing the above description. The scope of the inventive subject matter should, therefore, be determined with reference to the appended claims, along with the full scope of equivalents to which such claims may be entitled. In the appended claims, the terms “including” and “in which” may be used as the plain-English equivalents of the respective terms “comprising” and “wherein.” Moreover, in the following claims, the terms “first,” “second,” and “third,” etc. may be used merely as labels, and may be not intended to impose numerical requirements on their objects.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims is intended to be a means-plus-function element within the meaning of 35 U.S.C. § 112 (f) unless an element is expressly recited using the phrase “means for,” or in the case of a method claim using the phrases “operation for” or “step for.”

The methods described herein do not have to be executed in the order described, or in any particular order. Moreover, various activities described with respect to the methods identified herein can be executed in serial or parallel fashion. Although “End” blocks may be shown in the flowcharts, the methods may be performed continuously.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

What is claimed is:

1. An interactive patient monitoring system, the monitoring system comprising:
a robotic apparatus including physical characteristics representative of an animal or other companion with whom a human could interact, the robotic apparatus including one or more actuators capable of causing the robotic apparatus to perform one or more actions;
a communication device configured to communicate with a sensing device to receive measurements of one or more medical characteristics of a patient; and
a controller configured to examine the measurements received from the sensing device and to control the one or more actuators of the robotic apparatus to interact with the patient based on the measurements received from the sensing device.

2. The monitoring system of claim 1, wherein the controller is configured to control the one or more actuators to move the robotic apparatus to interact with the patient.

3. The monitoring system of claim 1, wherein the controller is configured to control the one or more actuators to generate light, generate one or more sounds, or place a telephone call by the robotic apparatus.

4. The monitoring system of claim 1, wherein the robotic apparatus has a shape of one or more of a dog, a cat, or a bird.

5. The monitoring system of claim 1, wherein the communication device is configured to receive the measurements from the sensing device as blood glucose measurements of a blood glucose of the patient as the one or more medical characteristics of the patient.

6. The monitoring system of claim 5, wherein the controller is configured to control the one or more actuators to cause the robotic apparatus to generate one or more sounds responsive to the blood glucose measurements indicating or trending toward a hypoglycemic condition of the patient.

7. The monitoring system of claim 5, wherein the controller is configured to control the communication device to cause the communication device to send a wireless notification to a third party responsive to the blood glucose measurements indicating or trending toward a hypoglycemic condition of the patient.

8. The monitoring system of claim 5, wherein the controller is configured to control the one or more actuators to cause the robotic apparatus to move and indicate that the patient should increase an activity level of the patient responsive to the blood glucose measurements indicating or trending toward a hyperglycemic condition of the patient.

9. The monitoring system of claim 1, wherein the controller is configured to control one or more of the communication device or the one or more actuators to direct the patient, via the robotic apparatus, to perform a blood glucose measurement of the patient.

10. The monitoring system of claim 1, wherein the controller includes or is part of a machine learning or artificial intelligence neural network configured to examine the measurements received from the sensing device using a model that defines relationships between different ones of the one or more actions of the robotic apparatus and different values or trends of the measurements, the controller configured to select one or more of the different ones of the one or more actions and to control the one or more actuators to implement the one or more of the different ones of the one or more actions that is selected using the model.

11. The monitoring system of claim 1, further comprising an acoustic output device configured to be disposed in or on the robotic apparatus and to generate different sounds, the controller configured to select one or more of the different sounds to be played by the acoustic output device based on the measurements received from the sensing device.

12. The monitoring system of claim 11, wherein the different sounds represent voices of different persons.

13. The monitoring system of claim 11, further comprising an acoustic input device configured to be disposed in or on the robotic apparatus and to detect a voice of the patient, the controller configured to control the acoustic input device, the acoustic output device, and the communication device to audibly connect the patient with a third party via the robotic apparatus.

14. The monitoring system of claim 1, wherein one or more actuators of the robotic apparatus include one or more of a lamp configured to generate light or a motor configured to generate vibrations of the robotic apparatus based on the measurements that are received by the controller from the one or more sensing devices.

15. The monitoring system of claim 1, wherein the controller is configured to communicate with one or more external devices that are external to the robotic apparatus to one or more of communicate one or more of the measurements or to control the one or more external devices based on the measurements that are received.

16. The monitoring system of claim 1, wherein the measurements received from the sensing device are first measurements, and further comprising an internal sensor of the robotic apparatus configured to sense second measurements of the one or more medical characteristics of the patient, the controller configured to control the one or more actuators of the robotic apparatus based on the second measurements obtained from the internal sensor.

17. The monitoring system of claim 1, wherein the robotic apparatus has a shape of an animal pet, the controller is configured to receive blood glucose measurements from the sensing device as the measurements, the controller is configured to control the one or more actuators to cause the robotic apparatus to generate an animal sound or move responsive to the blood glucose measurements indicating a hypoglycemic state of the patient or a trend toward the hypoglycemic state of the patient, the controller configured to control the one or more actuators to cause the robotic apparatus to prompt the patient to increase activity responsive to the blood glucose measurements indicating a hyperglycemic state of the patient or a trend toward the hyperglycemic state of the patient.

18. A method comprising:
receiving measurements of one or more medical characteristics of a patient from a sensing device, the measurements received at a communication device of a robotic apparatus;
identifying a medical state based on the measurements that are received; and
controlling one or more actuators of the robotic apparatus to cause the robotic apparatus to interact with the patient based on the medical state that is identified.

19. The method of claim 18, wherein controlling the one or more actuators includes one or more of:
generating one or more sounds with the robotic apparatus responsive to the measurements indicating or trending toward a hypoglycemic condition of the patient; or moving the robotic apparatus to indicate that the patient should increase an activity level of the patient responsive to the measurements indicating or trending toward a hyperglycemic condition of the patient.

20. An interactive patient monitoring system, the monitoring system comprising:
a robotic apparatus having a shape of an animal pet, the robotic apparatus comprising one or more actuators configured to simulate movement of the animal pet by moving the robotic apparatus;
a communication device configured to communicate with a continuous glucose measurement (CGM) device to receive measurements of blood glucose levels of a patient; and
a controller configured to examine the measurements received from the CGM device and to control the one or more actuators of the robotic apparatus to simulate movement of the animal pet and interact with the patient in different ways based on the blood glucose measurements received from the CGM device.
